SEQUENCE LISTING (2 sequences)

SEQ ID NO: 1 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - RNA, 2015 nt, Homo sapiens
  source 1..2015: mol_type = mRNA
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttga

(19) United States
(12) Patent Application Publication
(10) Pub. No.: US 2025/0298015 A1
(43) Pub. Date: Sep. 25, 2025

(54) DIAGNOSTIC METHOD OF DETECTING INFLAMMATION BIOMARKER(S)

(71) Applicant: RNA Care Co Limited, Dunedin (NZ)

(72) Inventors: Libby Lindsay, Dunedin (NZ); Harry Banaharis, Dunedin (NZ)

(73) Assignee: RNA Care Co Limited, Dunedin (NZ)

(21) Appl. No.: 19/085,233

(22) Filed: Mar. 20, 2025

(30) Foreign Application Priority Data
Mar. 21, 2024 (NZ) 809400

(51) Int. Cl. G01N 33/543

(52) U.S. Cl. CPC G01N 33/54388; G01N 2333/4737; G01N 2800/7095

(57) ABSTRACT

The present invention relates to inflammation, in particular methods of detecting inflammation, measuring or predicting levels of inflammation, predicting the likely or determining the actual responsiveness to treatment and/or treating, preventing or ameliorating inflammation or a disease or condition associated with inflammation.

### FIELD

The present invention relates to inflammation, and in particular methods of detecting inflammation, measuring or predicting levels of inflammation, predicting the likely or determining the actual responsiveness to treatment and/or treating, preventing or ameliorating inflammation or a disease or condition associated with inflammation. However, it will be appreciated that the invention is not limited to this particular field of use.

### BACKGROUND

Inflammation is part of a complex response of the innate immune system to harmful stimuli, such as pathogens, infection, irritants, or damage to cells. The inflammatory response is a defence mechanism to protect the organism by localising and eliminating the harmful stimuli and to remove the damaged tissue components so that the body can begin to heal.

Inflammation can be classified as either acute or chronic. Acute inflammation is the initial response to harmful stimuli that is mediated by plasma and leukocytes (in particular granulocytes), while chronic inflammation is a slower and generally less severe form of inflammation that leads to a progressive shift of mononuclear cells such as monocytes and lymphocytes to the site of inflammation for simultaneous destruction and healing of tissue. Chronic inflammation has been linked to a wide range of seemingly unrelated disorders and diseases such as cardiovascular diseases, cancers, allergies, obesity, diabetes, digestive system diseases, degenerative diseases, auto-immune disorders, and Alzheimer's disease. Chronic inflammation can also be caused by prolonged stress as it can occur even when there is no injury, and it does not always stop when the illness or injury is healed. The five cardinal signs of acute inflammation are heat, pain, redness, swelling, and loss of function (InformedHealth.org; 2006), whereas the common signs and symptoms that develop during chronic inflammation are body pain, arthralgia, myalgia, chronic fatigue and insomnia, depression, anxiety and mood disorders, gastrointestinal complications such as constipation, diarrhea, and acid reflux, weight gain or loss, and frequent infections.

Methods for detecting and measuring level of inflammation is limited and are conventionally done via a blood test. The most broadly used blood serum protein biomarkers for initiating investigations into inflammation are erythrocyte sedimentation rate (ESR), C-reactive protein (CRP) and plasma viscosity (PV) (Zimmerman, Selzman et al. 2003, Germolec, Shipkowski et al. 2018, Watson, Jones et al. 2019).

CRP levels in blood are associated with systemic inflammation due to injury, infection or various disease processes. CRP is not merely a marker of inflammation but is an active mediator. In one well characterised mechanism, CRP has been shown to bind to cell membranes of bacteria, host dying or dead cells. This activates the complement system, which in turn promotes their removal via macrophages. CRP is considered a central regulator of the innate immune response.

The normal CRP range is <3 mg/L while inflammatory conditions can drive the value many 100× over. CRP is measured either in the range of 10-1,000 mg/L for infections or as high-sensitivity CRP (hsCRP) in the range of 0.5-10 mg/L for chronic conditions. Chronic CRP levels over >3 mg/L can be caused by increased BMI, smoking, type 2 diabetes, estrogen use or autoimmune conditions and therefore highly amenable to dietary and lifestyle interventions.

Indeed low-grade inflammation is involved in many chronic diseases, which is consistent with the extent of disease phenotypes associated with CRP (Markozannes, Koutsioumpa et al. 2021).

High levels of CRP are associated with more severe forms of COVID-19 (Luan, Yin et al. 2021) and a CRP>200 mg/L on admission has a stronger association with adverse outcomes than age or comorbidities by a factor of 5 (Cihakova, Streiff et al. 2021).

Conventional blood test to measure proteins such as ESR, CRP and PV requires drawing blood from the patient is an invasive procedure and can be inconvenient and undesirable to the patient and/or healthcare provider. Such an invasive procedure can be intimidating for patients and can lead to pain, discomfort or stress to the patient. Attending a laboratory or a medical facility for drawing blood can also be inconvenient for the patient. For healthcare providers such as those in the field of nutrigenomics, it is not practical or cost-effective to collect blood samples on all potential patients or customers.

### SUMMARY OF THE INVENTION

The present invention broadly relates to methods of detecting and/or measuring or predicting levels of inflammation, predicting the likely, or determining the actual, responsiveness to treatment of inflammation or a disease or condition associated with inflammation and or treating, preventing or ameliorating inflammation or a disease or condition associated with inflammation which overcome or ameliorate at least one of the disadvantages of the prior art, or to provide a useful alternative. In one or more embodiments, the present invention relates to detecting level of CRP ribonucleic acid (RNA) obtained from a buccal cavity as a biomarker for inflammation.

According to a first embodiment of the invention there is provided a method for detecting at least one biomarker in a subject comprising:
a) assaying a sample obtained from the subject, and
b) detecting the at least one biomarker from the sample,
   wherein the sample is obtained from a buccal cavity of the subject and wherein the at least one biomarker is C-reactive protein (CRP) ribonucleic acid (RNA).

Preferably, the subject has suspected to have, or is recovering from, inflammation.

According to a second embodiment of the invention, there is provided a method of predicting a level of inflammation in a subject comprising:
a) assaying a sample obtained from the subject, and
b) detecting at least one biomarker from the biological sample, wherein the sample is obtained from a buccal cavity of the subject and wherein the at least one biomarker is CRP RNA.

The RNA preferably is a messenger ribonucleic acid (mRNA).

In one aspect, the sample is a biological sample selected from saliva or sputum. In another aspect, the sample is a buccal cavity swab.

Preferably, the method further comprises correlating the concentration of the at least one biomarker from the sample of the subject with at least one predetermined reference concentration of the at least one biomarker.

Preferably, the method further comprises assigning an inflammation level based on the correlation. Preferred embodiments of this approach will be discussed in the Detailed Description.

In one aspect, the at least one predetermined reference concentration of the at least one biomarker is from a sample obtained from a healthy subject. In another aspect, the at least one predetermined reference concentration of the at least one biomarker is from a sample obtained from an unhealthy or diseased subject.

In another aspect, heat least one predetermined reference concentration of the at least one biomarker is from a blood sample obtained from the same or different subject, for example a plasma CRP protein concentration measured from the same or different subject. The inventors envisage that a reference standard from multiple subjects (using both predetermined reference concentration of plasma CRP levels against the RNA biomarker from the same subjects). This may be developed to enable efficient future testing of subjects via just a RNA biomarker, and then correlating that to a predicted plasma CRP level and hence a prediction for level of inflammation of that subject, without needing to take blood samples.

According to a third embodiment of the invention, there is provided a method of predicting a level of inflammation level in a subject comprising:
a) assaying a sample obtained from the subject,
b) detecting at least one biomarker from the sample,
c) determining the concentration of the at least one biomarker from the sample and comparing with at least one predetermined reference concentration of the at least one biomarker, and
d) assigning the predicted level of inflammation based on the comparison,
   wherein the sample is obtained from a buccal cavity of the subject and wherein the at least one biomarker is CRP RNA.

Preferably, the at least one predetermined reference concentration is <1.0 mg/L, 1.0-3.0 mg/L, and/or >3.0 mg/L.

Preferably, the inflammation is low grade inflammation, acute inflammation or chronic inflammation.

In one aspect, the inflammation level is a low-grade inflammation. Preferably, the at least one predetermined reference concentration is between 3-10 mg/L.

In another aspect, the inflammation level is acute inflammation or chronic inflammation.

Preferably, the at least one predetermined concentration is above 10 mg/L.

In one aspect, the at least one biomarker is detected by a lateral flow assay.

In one aspect, the concentration of the at least one biomarker from the sample is determined by a lateral flow assay.

According to a fourth embodiment of the invention, there is provided a method of determining the responsiveness of a subject to treatment or prevention of inflammation, or a disease or condition associated with inflammation comprising:
a) assaying a biological sample obtained from the subject,
b) detecting at least one biomarker from the biological sample,
c) determining the concentration of the at least one biomarker from the sample, and
d) comparing with at least one predetermined reference concentration of the at least one biomarker,
   wherein the biological sample is obtained from a buccal cavity of the subject, wherein the at least one biomarker is CRP RNA, and wherein the concentration of the at least one biomarker from the sample indicates or correlates with relatively increased or decreased responsiveness of the subject to the treatment or prevention of inflammation.

In one aspect, a decreased concentration of the at least one biomarker from the sample indicates or correlates with relatively increased responsiveness of the subject to the treatment or prevention of inflammation. In another aspect, an increased concentration of the at least one biomarker from the sample indicates or correlates with relatively decreased responsiveness of the subject to the treatment or prevention of inflammation.

In one aspect, the at least one biomarker is detected by a lateral flow assay.

In one aspect, the concentration of the at least one biomarker from the sample is determined by a lateral flow assay.

According to a fifth embodiment of the invention, there is provided a method of treating, preventing or ameliorating inflammation in a subject comprising:
detecting or measuring CRP RNA levels according to the first, second or third embodiment, and
recommending, making, initiating, continuing, modifying or discontinuing a treatment to the subject.

According to a sixth embodiment of the invention, there is provided a method of treating, preventing or ameliorating at least one disease or condition associated with inflammation in a subject comprising:
detecting or measuring CRP RNA levels according to the first, second or third embodiment, and
recommending, making, initiating, continuing, modifying or discontinuing a treatment to the subject.

In one aspect, the treatment comprises administering an effective amount of the treatment to the subject.

Preferably, the treatment is selected from one or more of a food, a nutrient, a drug, an agent, and a lifestyle change or plan.

In one aspect, the condition associated with inflammation is selected from one or more of redness, swelling, heat, pain, and loss of tissue function. In another aspect, disease associated with inflammation is a cardiovascular disease, bowel diseases, diabetes, arthritis, pancreatitis, liver disease, lung inflammatory disease, kidney disease, intestinal tract disease, brain disease, or cancer.

According to a seventh embodiment of the invention, there is provided a kit for use in the method according to the first, second or third embodiment, comprising:
means for collecting the sample from the subject, and
a composition for stabilising the sample obtained from the subject.

According to an eighth embodiment of the invention, there is provided a composition for stabilising a sample for use in the method according to any one of the first to fifth embodiment.

According to a ninth embodiment of the invention, there is provided a composition for detecting at least one biomarker in a sample obtained from a subject comprising a molecule that binds to the at least one biomarker, wherein the sample is obtained from a buccal cavity of the subject and wherein the at least one biomarker is CRPRNA.

The indefinite articles ‘a’ and ‘an’ are used here to refer to or encompass singular or plural elements or features and should not be taken as meaning or defining “one” or a “single” element or feature. For example, “a” cell includes one cell, one or more cells and a plurality of cells.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

### DETAILED DESCRIPTION

In one or more embodiments, the present invention is at least partly predicated on providing a useful biomarker for inflammation as a less invasive approach to the conventional blood test.

In addition, in one or more embodiments the invention is at least partly predicated on detecting levels of CRP mRNA from cells in the buccal cavity such as from a saliva sample or a buccal cavity swab (e.g., oral CRP mRNA) surprisingly offering a reliable correlation to the CRP concentration in blood. Further, in one or more embodiments the present invention is at least partly predicated on a method, a composition or a kit of preserving buccal cavity samples which is generally difficult as any stressing of the cells being taken for sampling can lead to changes in mRNA transcription, and therefore could inaccurately represent physiological levels of the CRP protein levels.

Accordingly, in a first embodiment of the invention there is provided a method for detecting at least one biomarker in a subject comprising:
a) assaying a sample obtained from the subject, and
b) detecting the at least one biomarker from the sample,
   wherein the sample is obtained from a buccal cavity of the subject and wherein the at least one biomarker is C-reactive protein (CRP) ribonucleic acid (RNA).

Preferably, the subject has, suspected to have or is recovering from inflammation.

In a second embodiment of the invention there is provided a method of predicting a level of inflammation in a subject comprising:
a) assaying a sample obtained from the subject, and
b) detecting at least one biomarker from the sample,
wherein the sample is obtained from a buccal cavity of the subject and wherein the at least one biomarker is CRP RNA.

As generally used herein, “CRP” refers to C-reactive protein. It should be understood that the protein sequence, structure and function of this CRP protein may vary depending on natural or induced changes found in the general population and resulting from genetic differences as discussed further below. The amino acid sequence of CRP is shown in SEQ ID No. 1.

(SEQ ID NO. 1)
MEKLLCFLVLTSLSHAFGQTDMSRKAFVFPKESDTSYVSLKAPLTKPL
KAFTVCLHFYTELSSTRGYSIFSYATKRQDNEILIFWSKDIGYSFTVG
GSEILFEVPEVTVAPVHICTSWESASGIVEFWVDGKPRVRKSLKKGYT
VGAEASIILGQEQDSFGGNFEGSQSLVGDIGNVNMWDFVLSPDEINTI
YLGGPFSPNVLNWRALKYEVQGEVFTKPQLWP

While the CRP gene is primarily expressed in liver hepatocytes (and therefore has been the conventional approach to measuring CRP in blood plasma), extrahepatic synthesis of CRP has been documented in neurons, atherosclerotic plaques, monocytes, lymphocytes, macrophages, smooth muscle cells, endothelial cells and adipocytes. Regardless, to the best of the inventor's knowledge, these CRP sources have not been investigated or used for biomarkers because they are not the most abundant and therefore reliable source of CRP determination, and little is known about their correlation with hepatic synthesis.

The CRP gene is located on the short arm of chromosome 1. Its promoter has binding sites for the chemokines, interleukin 6 (IL6) and interleukin 1 beta (IL1b), transcription factors STAT3 and two separate sites for C/EBPb. IL6 is considered the most important regulator for hepatic CRP expression.

Although expression of CRP from non-hepatic sources does not significantly contribute to systemic CRP as detected in blood collections, its activation by systemic chemokines suggests that non-hepatic CRP may be contemporaneously expressed in parallel with hepatic CRP. Therefore, it was envisaged by the inventors that the signalling messenger ribonucleic acid (mRNA) from such cells may potentially be useful as mRNA CRP biomarkers from sources other than blood. Despite this, there was still considerable uncertainty whether the level of non-hepatic CRP mRNA obtained from alternative sources such as oral samples would or could be used as a viable biomarker and predictor of actual CRP levels in blood, for example from circulating liver hepatocytes. Furthermore, the complexities and instability of mRNA as a biomarker for CRP added even further ambiguity and uncertainty around its usefulness as a predictive tool for circulating CRP.

Accordingly, the RNA preferably is mRNA.

RNA biomarkers are concerned with changes to the amount of a specific RNA produced (i.e., transcript) within the same individual and offer early and more accurate prediction and diagnosis of disease and disease progression, and ability to identify individuals at risk. Specifically, RNA biomarkers have more sensitivity and specificity as they describe (i) the type and (ii) the amount of transcript.

While each cell in the human body carries a (mostly) identical copy of the genome, depending on its functional role in the body, each cell, tissue and organ may express an entirely different transcriptome. Moreover, that transcriptome changes with age and health. Accordingly, the type and amount of RNA transcripts under go changes and are responsive to external factors such as diet and environmental exposures.

Despite the complexities and unpredictability of sampling an RNA biomarker from alternative sources, the dynamic nature of RNA suggested to the inventors of the potential benefits and utility as a biomarker related to changing physiological parameters (analogous to biochemical analytes measured in blood collections). In addition, the amount of mRNA transcribed from a gene can be related to the amount of protein translated from that mRNA. Therefore, mRNA quantification was considered by the inventors as a potential surrogate marker for CRP's associated protein product, with considerable advantages over the latter conventional approach to detecting/predicting levels of inflammation.

As used herein a “gene” is a nucleic acid which is a structural, genetic unit of a genome that may include one or more amino acid-encoding nucleotide sequences and one or more non-coding nucleotide sequences inclusive of promoters and other 5′ untranslated sequences, introns, polyadenylation sequences and other 3′ untranslated sequences, although without limitation thereto. In most cellular organisms a gene is a nucleic acid that comprises double-stranded DNA

The term “nucleic acid” as used herein designates single- or double-stranded DNA and RNA. DNA includes genomic DNA and cDNA. RNA includes mRNA, RNA, RNAi, siRNA, cRNA, miRNA and autocatalytic RNA. Nucleic acids may also be DNA/RNA hybrids. A nucleic acid comprises a nucleotide sequence which typically includes nucleotides that comprise an A, G, C, T or U base. However, nucleotide sequences may include other bases such as inosine, methylcytosine, methylinosine, methyladenosine and/or thiouridine, although without limitation thereto.

As used herein, “messenger RNA” (“mRNA”) is a single stranded RNA that corresponds to the genetic sequence of a gene and is read by a ribosome in the process of synthesizing a protein. The preferred mRNA sequence for CRP is shown in SEQ ID No. 2.

| (SEQ ID NO. 2) | |
|---|---|
| 1 | aaggcaagag atctaggact tctagcccct gaactttcag ccgaatacat cttttccaaa |
| 61 | ggagtgaatt caggcccttg tatcactggc agcaggacgt gaccatggag aagctgttgt |
| 121 | gtttcttggt cttgaccagc ctctctcatg cttttggcca gacagacatg tcgaggaagg |
| 181 | cttttgtgtt tcccaaagag tcggatactt cctatgtatc cctcaaagca ccgttaacga |
| 241 | agcctctcaa agccttcact gtgtgcctcc acttctacac ggaactgtcc tcgacccgtg |
| 301 | ggtacagtat tttctcgtat gccaccaaga gacaagacaa tgagattctc atattttggt |
| 361 | ctaaggatat aggatacagt tttacagtgg gtgggtctga aatattattc gaggttcctg |
| 421 | aagtcacagt agctccagta cacatttgta caagctggga gtccgcctca gggatcgtgg |
| 481 | agttctgggt agatgggaag cccagggtga ggaagagtct gaagaaggga tacactgtgg |
| 541 | gggcagaagc aagcatcatc ttggggcagg agcaggattc cttcggtggg aactttgaag |
| 601 | gaagccagtc cctggtggga gacattggaa atgtgaacat gtgggacttt gtgctgtcac |
| 661 | cagatgagat taacaccatc tatcttggcg ggcccttcag tcctaatgtc ctgaactggc |
| 721 | gggcactgaa gtatgaagtg caaggcgaag tgttcaccaa accccagctg tggccctgag |
| 781 | gcccagctgt gggtcctgaa ggtacctccc ggttttttac accgcatggg ccccacgtct |
| 841 | ctgtctctgg tacctcccgc ttttttacac tgcatggttc ccacgtctct gtctctgggc |
| 901 | ctttgttccc ctatatgcat tgcaggcctg ctccaccctc ctcagcgcct gagaatggag |
| 961 | gtaaagtgtc tggtctggga gctcgttaac tatgctggga aacggtccaa aagaatcaga |
| 1021 | atttgaggtg ttttgttttc atttttattt caagttggac agatcttgga gataatttct |
| 1081 | tacctcacat agatgagaaa actaacaccc agaaaggaga aatgatgtta taaaaaactc |
| 1141 | ataaggcaag agctgagaag gaagcgctga tcttctattt aattccccac ccatgacccc |
| 1201 | cagaaagcag gagggcattg cccacattca cagggctctt cagtctcaga atcaggacac |
| 1261 | tggccaggtg tctggtttgg gtccagagtg ctcatcatca tgtcatagaa ctgctgggcc |
| 1321 | caggtctcct gaaatgggaa gcccagcaat accacgcagt ccctccactt tctcaaagca |
| 1381 | cactggaaag gccattagaa ttgccccagc agagcagatc tgcttttttt ccagagcaaa |
| 1441 | atgaagcact aggtataaat atgttgttac tgccaagaac ttaaatgact ggtttttgtt |
| 1501 | tgcttgcagt gctttcttaa ttttatggct cttctgggaa actcctcccc ttttccacac |
| 1561 | gaaccttgtg gggctgtgaa ttctttcttc atccccgcat tcccaatata cccaggccac |
| 1621 | aagagtggac gtgaaccaca gggtgtcctg tcagaggagc ccatctccca tctccccagc |
| 1681 | tccctatctg gaggatagtt ggatagttac gtgttcctag caggaccaac tacagtcttc |
| 1741 | ccaaggattg agttatggac tttgggagtg agacatcttc ttgctgctgg atttccaagc |
| 1801 | tgagaggacg tgaacctggg accaccagta gccatcttgt ttgccacatg gagagagact |
| 1861 | gtgaggacag aagccaaact ggaagtggag gagccaaggg attgacaaac aacagagcct |
| 1921 | tgaccacgtg gagtctctga atcagccttg tctggaacca gatctacacc tggactgccc |
| 1981 | aggtctataa gccaataaag cccctgttta cttga |

Also envisaged are “variant” nucleic acids that include nucleic acids that comprise nucleotide sequences of naturally occurring (e.g., allelic) variants and orthologs (e.g., from a different species) encoding CRP. Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. A non-limiting example is an amplification productor a primer or probe. In particular embodiments, a nucleic acid fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000 and 7500 contiguous nucleotides of said nucleic acid.

As used herein, a “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

A “template” nucleic acid is a nucleic acid subjected to nucleic acid amplification.

The term “sample” is used herein to refer to a sample that is extracted from a subject. The term encompasses untreated, treated, diluted or concentrated biological samples. The sample obtained from the subject can be any suitable sample, such as whole blood serum plasma, saliva, sputum, or buccal sample (e.g., cells from the buccal cavity collected from a buccal cavity swab).

Preferably, the sample is obtained from the buccal cavity of the subject. In one aspect, the sample is a biological sample selected from saliva or sputum. In another aspect, the sample is a buccal cavity swab.

Expression of a gene isa function of its cell type as well as many other factors that regulate its activity. Unlike the case for DNA where every cell is representative of the entire genome, RNA transcripts are strongly tissue and cell specific. Genetic samples may be obtained from oral collections and are therefore constrained to the gene expression profiles available from the cells present in such collections. Beneficially, saliva collections include epithelial cells and leukocytes, whereas buccal swabs are biased towards epithelial cells but also include leukocytes.

Therefore, the inventors envisage that, for the purpose as an inflammatory biomarker, the leukocyte cell population, which includes lymphocytes, neutrophils, eosinophils and mast cells may therefore be a suitable representative of CRP gene expression related to a significant subset of immunological function. Notably, conditions like gingivitis may cause the proportion of leukocytes to increase significantly, but given transcripts can be tested using an internal housekeeping gene control, the altered proportion of leukocyte to epithelial cells should not affect the measurement accuracy of changes to transcript levels.

Preferably, the method further comprises correlating the concentration of the at least one biomarker from the sample of the subject with at least one predetermined reference concentration of the at least one biomarker. Preferably, the method further comprises assigning an inflammation level based on the correlation.

In a third embodiment of the invention there is provided a method of predicting a level of inflammation level in a subject comprising:
a) assaying a sample obtained from the subject,
b) detecting at least one biomarker from the sample,
c) determining the concentration of the at least one biomarker from the sample and comparing with at least one predetermined reference concentration of the at least one biomarker, and
d) assigning the predicted level of inflammation based on the comparison,
wherein the sample is obtained from a buccal cavity of the subject and wherein the at least one biomarker is CRP RNA.

As used herein, the “inflammation level” is preferably selected from low grade inflammation, acute inflammation or chronic inflammation.

As would be understood by the skilled person, the concentration level of the CRP biomarker from the subject may be relatively (i) higher, increased or greater; or (ii) lower, decreased or reduced when compared to a concentration level in a control or reference sample, or to a threshold concentration level. The terms “control sample”, “reference sample” and “threshold sample” are used interchangeably herein and may include the predetermined reference sample.

In one embodiment, a CRP level may be classified as higher, increased or greater if it exceeds a mean and/or median expression level of a reference population. Alternatively, a CRP level may be classified as lower, decreased or reduced if it is less than the mean and/or median expression level of the reference population. In this regard, a reference sample may be obtained from a healthy subject, a diseased subject, a previous collection of the same subject, or a sample collected from the same subject from a different site (e.g., a plasma CRP protein concentration measured from the same subject). A reference population may be a group of healthy subjects or diseased subjects for which the expression level is determined.

Similarly, it is envisaged that a reference or standard correlating mRNA encoding CRP and actual CRP expression from a range of subjects from healthy/low inflammation to unhealthy/high inflammation may be developed such that future predictions of inflammation, and suitable interventions, may be made based on the predetermined reference or standard.

In one embodiment, the concentration level of the CRP biomarker from the subject may be classified as (i) high or (ii) low when it falls within a predetermined reference or standard concentration level or range.

For example, a predetermined reference concentration level or range of CRP (e.g., CRP protein) is <1.0 mg/L (no CRP activation), 1.0-3.0 mg/L (medium CRP activation), and >3.0 mg/L (High CRP Activation). In one example, a predetermined reference concentration level or range of CRP (e.g., CRP protein) is between 3-10 mg/L. In another example, a predetermined reference concentration level or range of CRP (e.g., CRP protein) is above 10 mg/L.

In an alternative embodiment, the predicted inflammation level is a low-grade inflammation wherein the at least one predetermined reference concentration is between 3-10 mg/L.

In another aspect, the predicted inflammation level is acute inflammation or chronic inflammation wherein the at least one predetermined concentration is above 10 mg/L.

Terms such as “higher”, “increased” and “greater” as used herein refer to an elevated amount or level of a CRP nucleic acid or protein, such as in a biological sample, when compared to a control or reference level or amount. The concentration level of the CRP nucleic acid or protein may be relative or absolute. In some embodiments, the concentration of the CRP nucleic acid or protein is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of concentration of a CRP nucleic acid or protein in a control or reference level or amount.

The terms, “lower”, “reduced” and “decreased”, as used herein refer to a lower amount or level of the CRP nucleic acid or protein, such as in a biological sample, when compared to a control or reference level or amount. The concentration level of the CRP nucleic acid or protein may be relative or absolute. In some embodiments, the expression of the CRP nucleic acid or protein is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of concentration of the CRP nucleic acid or protein in a control or reference level or amount.

The term “control sample” typically refers to a biological sample from a (healthy) non-diseased individual or a diseased individual. The control sample may also be a sample from a previous collection of the same subject, or a sample collected from the same subject from a different site (e.g., a CRP protein concentration or level detected in a blood sample). The control sample may be a pooled, average or an individual sample. An internal control is a marker from the same biological sample being tested.

As used herein, a concentration level may be an absolute or relative amount of a nucleic acid (such as transcribed mRNA) or expressed protein. Accordingly, in some embodiments, the concentration level of the CRP nucleic acid and/or a product thereof is compared to a control level of concentration, such as the level of nucleic acid and/or protein concentration of one or a plurality of“housekeeping” genes and/or proteins in one or more cells, tissues or organs of the subject.

In further embodiments, the concentration level of the CRP nucleic acid or encoded protein is compared to a threshold level of concentration, such as a level of nucleic acid and/or protein concentration in a sample from a healthy subject or population or a diseased subject or population. A threshold level of concentration is generally a quantified level of concentration of CRP. Typically, a concentration level of CRP in a sample that exceeds or falls below the threshold level of expression is predictive of a particular disease state or outcome. The nature and numerical value (if any) of the threshold level of concentration will typically vary based on the method chosen to determine the concentration of the one or more nucleic acid, or products thereof, used in determining, for example, a prognosis and/or a response to a treatment of inflammation or a disease or condition associated with inflammation in the subject.

A person of skill in the art would be capable of determining the threshold level of CRP nucleic acid or protein concentration in a sample that may be used in determining, for example, a prognosis and/or a response to a treatment of inflammation or a disease or condition associated with inflammation, using any method of measuring nucleic acid or protein concentration known in the art, such as those described herein. In one embodiment, the threshold level is a mean and/or median concentration level (median or absolute) of CRP in a reference population, that, for example, have the same inflammation level and/or a disease or condition associated with inflammation as said subject for which the concentration level is determined. Additionally, the concept of a threshold level of concentration should not be limited to a single value or result. In this regard, a threshold level of concentration may encompass multiple threshold expression levels that could signify, for example, a high, medium, or low probability of, for example, response to treatment or prevention of inflammation.

Accordingly, in a fourth embodiment of the invention there is provided a method of determining the responsiveness of a subject to treatment or prevention of inflammation, or a disease or condition associated with inflammation comprising:
a) assaying a biological sample obtained from the subject,
b) detecting at least one biomarker from the biological sample,
c) determining the concentration of the at least one biomarker from the sample, and
d) comparing with at least one predetermined reference concentration of the at least one biomarker,
wherein the biological sample is obtained from a buccal cavity of the subject, wherein the at least one biomarker is CRP RNA, and wherein the concentration of the at least one biomarker from the sample indicates or correlates with relatively increased or decreased responsiveness of the subject to the treatment or prevention of inflammation.

Suitably, a reduced, decreased or lower concentration level of CRP nucleic acid or encoded protein indicates or correlates with relatively increased responsiveness of the subject to the treatment or prevention of inflammation. Conversely, a greater, increased or higher expression or concentration level of CRP nucleic acid or encoded protein may indicate or correlate with relatively decreased responsiveness of the subject to the treatment or prevention of inflammation. In this regard, the CRP concentration levels are useful in the prediction of sensitivity and/or resistance of the subject's body to the treatment or prevention of inflammation.

The terms “determining”, “measuring”, “evaluating”, “assessing” and “assaying” are used interchangeably herein and may include any form of measurement known in the art, such as those described hereinafter.

Determining, assessing, evaluating, assaying or measuring nucleic acids of CRP, such as RNA, mRNA and cDNA, may be performed by any technique known in the art. These may be techniques that include nucleic acid sequence amplification, nucleic acid hybridization, nucleotide sequencing, mass spectroscopy, lateral flow assay and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence.

Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-β replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, reverse transcription PCR (RT-PCR), allele specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a cell or tissue source. In other embodiments, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

A significant challenge faced by the inventors was keeping the RNA molecule intact until it could become converted to cDNA (see below). Unlike DNA, which is a relatively stable molecule due to its double stranded helical structure, RNA molecules are single stranded and mRNA in particular is more sensitive to breakage. In addition to the standard nucleic acid quality control measures of spectrophotometric analysis that confirm purity, an RNA integrity number (RIN) should also be obtained to confirm that degradation has not occurred to a level that will compromise downstream analysis.

Aside from additional care during initial handling the isolation of RNA is much like DNA: disruption of cell membranes to access RNA, removal of proteins to prevent damage to RNA and isolation of RNA from other cell material. mRNA is distinguished from other RNA molecules due to the presence of a long sequence of Adenine (A) bases on one end (polyA tail). This characteristic enables it to be selectively targeted for purification. Once isolated, mRNA is converted to complementary DNA (cDNA) using a retro-transcriptase enzyme and then analysed using techniques associated with DNA analysis.

To measure the number of RNA transcripts present for a target gene, the analytic methodology is very similar to the analysis of DNA copy number variations, i.e. the number of copies of cDNA is measured and compared against an internal control cDNA derived from the transcript of a house keeping gene.

There are two main technologies currently in use to characterise mRNA transcripts:
1) Quantitative reverse transcription PCR (RT-qPCR): enables cDNA to be used as a template for the qPCR reaction to test for the activity from 1 to a few hundred genes using similar microarray methods as for SNP genotyping.
2) RNA sequencing (RNA-seq): a collection of methods analogous to next generation sequencing (NGS) that enable the massive parallelisation of cDNA analysis such that the activity of thousands of genes may be captured.

The main difference between RT-qPCR microarrays and RNA-Seq is that the latter allows for the sequencing of up to the entire transcriptome while the former only measures the activity of defined genes. For research purposes RNA-Seq is the superior technology but for biomarker purposes, where the target transcript has already been determined, RTqPCR microarrays are more suitable due to speed of analysis, precision and reproducibility of transcript quantification.

Lateral flow assay (LFA) is a well-known technique for the qualitative and quantitative detection of specific target substances (e.g., nucleic acid such as RNA, mRNA and cDNA) in liquid samples. It has been widely used in medical diagnostics, food safety and environmental monitoring (Koczula et al 2016, Posthuma-Trumpie et al. 2009, Quesada-Gonzdlez et al. 2015, and Sajid et al. 2015).

Devices based on LFA typically include a test strip comprising four main components: (i) a sample application pad, (ii) a conjugate pad, (iii) a nitrocellulose membrane and (iv) an adsorption pad. Various detection formats, molecular recognition probes, labels and detection systems can be employed in LFAs. In general, LFAs operate on the principle of capillary action, where a liquid sample flows along the test strip and activates pre-immobilized reagents and biorecognition molecules located at specific regions of the strip. The biorecognition molecules on the nitro cellulose membrane interact with the target substance and produce a visible result, typically in the form of coloured lines. Any molecules (e.g., protein, enzyme, oligonucleotides) that are known or specifically designed to detect, bind or interact with the target analytes can be used as the biorecognition molecules. Some examples of the biorecognition molecules include antibodies, aptamers and molecular beacons.

Antibodies have traditionally been used to detect specific proteins (e.g., antigens) in LFAs. They can also be utilized to detect nucleic acids such as RNA. For instance, antibodies have been developed to target RNA-binding proteins (RBP) that recognize specific RNA sequences or structures (https://www.thermofisher.com/au/en/home/references/newsletters-and-journals/bioprobes-journal-of-cell-biology-applications/bioprobes-80/rna-binding-protein-antibodies-validated-rna-immunopreciptation.html). Naturally occurring antibodies against RNA/protein (e.g., ribonucleoproteins) complexes are also present in autoimmune diseases such as systemic lupus erythematosus (SLE).

Aptamers are short, artificial single stranded nucleic acids that have very high association constants and can bind selectively with a variety of target analytes. They have been employed in selective chromatography, cell imaging, target capturing, in vivo therapy, molecular sensing, protein based imaging, cancer cell biology, as enzymes in many biological applications, cellular physiology, and drug delivery.

Molecular beacons are specialized DNA hairpin structure with a fluorophore at one end and a quencher at the other end (Sajid et al. 2015). They have been employed in messenger RNA detection, intercellular imaging, protein and small molecule analysis, biosensors, biochip development, single nucleotide polymorphism and gene expression studies.

Accordingly, in one aspect, the at least one biomarker is detected by a lateral flow assay. In one aspect, the concentration of the at least one biomarker from the sample is determined by a lateral flow assay.

Determining, assessing, evaluating, assaying or measuring protein levels of CRP may be performed by any technique known in the art that is capable of detecting cell- or tissue-expressed proteins whether on the cell surface or intracellularly expressed, or proteins that are isolated, extracted or otherwise obtained from the cell of tissue source. These techniques include antibody-based detection that uses one or more antibodies which bind the protein, electrophoresis, isoelectric focusing, protein sequencing, chromatographic techniques and mass spectroscopy and combinations of these, although without limitation thereto. Antibody-based detection may include flow cytometry using fluorescently-labelled antibodies that bind CRP, ELISA, immunoblotting, immunoprecipitation, in situ hybridization, immunohistochemistry and immunocytochemistry, although without limitation thereto.

Suitable techniques may be adapted for high throughput and/or rapid analysis such as using protein arrays such as a TissueMicroArray™ (TMA), MSD MultiArrays™ and multiwell ELISA, although without limitation thereto. It will be appreciated that determining the expression of CRP may include determining both the nucleic acid levels thereof, such as by nucleic acid amplification and/or nucleic acid hybridization, and the protein levels thereof.

In certain embodiments, a gene transcription level of CRP may be assessed indirectly by the measurement of a non-coding RNA, such as miRNA, that regulate gene expression. MicroRNAs (miRNAs or miRs) are post-transcriptional regulators that bind to complementary sequences in the 3′ untranslated regions (3′ UTRs) of target mRNA transcripts, usually resulting in gene silencing. miRNAs are short RNA molecules, on average only 22 nucleotides long. The human genome may encode over 1000 miRNAs, which may target about 60% of mammalian genes and are abundant in many human cell types. Each miRNA may alter the expression of hundreds of individual mRNAs. In particular, miRNAs may have multiple roles in negative regulation (e.g., transcript degradation and sequestering, translational suppression) and/or positive regulation (e.g., transcriptional and translational activation).

According to a fifth embodiment of the invention, there is provided a method of treating, preventing or a meliorating inflammation in a subject comprising:
detecting or measuring CRP RNA levels according to the first, second or third embodiment, and
recommending, making, initiating, continuing, modifying or discontinuing a treatment to the subject.

According to a sixth embodiment of the invention, there is provided a method of treating, preventing or ameliorating at least one disease or condition associated with inflammation in a subject comprising:

Detecting or measuring CRP RNA levels according to the first, second or third embodiment, and

recommending, making, initiating, continuing, modifying or discontinuing a treatment to the subject.

In one aspect, the treatment comprises administering an effective amount of the treatment to the subject.

As used herein, a “treatment” includes but not limited to a food, a nutrient, a drug, an agent, and a lifestyle change or plan.

According to a seventh embodiment of the invention, there is provided a kit for use in the method according to the first, second or third embodiment, comprising:
means for collecting the sample from the subject, and
a composition for stabilising the sample obtained from the subject.

According to an eighth embodiment of the invention, there is provided a composition for stabilising a sample for use in the method according to any one of the first to fifth embodiment.

According to a ninth embodiment of the invention, there is provided a composition for detecting at least one biomarker in a sample obtained from a subject comprising a molecule that binds to the at least one biomarker, wherein the sample is obtained from a buccal cavity of the subject and wherein the at least one biomarker is CRPRNA.

As used herein, a “condition associated with inflammation” includes but not limited to redness, swelling, heat, pain, and loss of tissue function. As used herein, a “disease associated with inflammation” includes but not limited to a cardiovascular disease, bowel diseases, diabetes, arthritis, pancreatitis, liver disease, lung inflammatory disease, kidney disease, intestinal tract disease, brain disease, or cancer.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). Preferably, the subject is a human. However it should be appreciated that the invention has application to other non-human animals. Merely for example, arthritis is a major problem in domesticated dogs as they age. Arthritis in dogs is triggered in part by the same inflammatory processes as previously discussed in humans, including increased expression of CRP, that contribute significantly to the progression of the disease. Therefore, the use of the methods employed within this application are applicable to non-human animals.

So that preferred embodiments of the invention may be fully understood and put into practical effect, reference is made to the following non-limiting examples.

### Example 1

### Sample Numbers

In order to establish the potential validity of CRP mRNA obtained from oral collections as a biomarker for systemic CRP (protein) a correlation would have to be demonstrated between oral CRP transcript levels and plasma CRP protein concentration.

A commonly used statistical test for determining the degree of linear association between two variables (i.e. CRP mRNA transcript number, and CRP plasma concentration) is Pearson's correlation coefficient. In order to perform the reverse, i.e. obtain the number of samples from a known correlation coefficient one may use the Fisher transformation. The Fisher transformation assumes a normal distribution of both variables and is therefore typically used as a guide for statistically informed sample size calculations.

A minimum sample size of 17 participants is used for a minimum acceptable linear correlation coefficient, of r=0.7, tolerance for a type I error rate (false positive) of 0.05 and type II error rate (false negative) of 0.10. In order to account for any dropouts or sample failures it would be appropriate to recruit 20 participants.

### Methods

### Sample Collection and Protection

A Buccal Swab can be obtained from a subject by the below steps:
(1) holding a swab handle and bring the swab tip (a sterile cotton, foam or flocked swab) to the inside of the subject's cheek,
(2) firmly rub and rotate the swab along the inside of the cheek for 5-10 seconds to ensure the entire swab tip has made contact with the subject's cheek,
(3) repeat step (2) on the other side of the mouth with the same swab,
(4) immediately remove the swab without touching the swab tip against the teeth, lips, or other surfaces,
(5) immediately place the swab in an aerated or air permeable container.

After collection, a commercially available RNA stabilizing solution can be added to the container and the swab can be stored at room temperature, at 4° C. or at −20° C. according to the instructions of the RNA stabilizing solution.

Extraction/Purifying mRNA from the Sample

To extract RNA from the buccal swab, the buccal swab tip is placed in a tube containing 200-400 μl of RNA lysis buffer (usually contain RNase inhibitors) that permeabilize the cell membrane to separate the cytoplasmic RNA from nuclear contends including gDNA. The tube is then vortex briefly and centrifuge for 2-10 minutes at 14,000-16,000×g to pellet the cell debris. The supernatant is then transferred to an RNase-free microfuge tube for further purification. Any available commercial RNA extraction and purification system such as the Promega® Maxwell 16 system with the Cell LEV Total purification kit can be used. The extracted RNA can be spectrophotometrically quantified (for example, by using the Thermo Fisher Nanodrop One Microvolume UV-Vis Spectrophotometer).

### RT-PCR

To carry out the two step RT-PCR, the extracted RNA is first transcribed into complementary DNA (cDNA). Any commercially available reverse transcription kit (e.g., Invitrogen Superscript 4 VILO Master Mix with ezDNase kit) can be used.

The cDNA is then used as the template for the quantitative PCR or real-time PCR reaction. CPR gene expression testing will be conducted using the Applied Biosystems Quantstudio 12 k system using real-time PCR with Taqman® Gene Expression Assay reagents Hs04183452_g1 for targeting the CRP gene and Hs02758991_g1 for the endogenous control gene GAPDH. In brief, the Taqman® Gene Expression Assay reagents include a forward primer and a reverse primer for the specific target gene (e.g., CRP gene or GAPDH) and one or more dye-labeled probes (e.g., fluorescently labeled probe) that specifically recognize and bind to the target gene during the PCR reaction. As the PCR reaction progresses, the TaqMan probe is cleaved by the Taq DNA polymerase enzyme to release the dye that results in a measurable signal.

Relative quantification of CRP gene expression is performed using the comparative Ct method, which involves comparing the Ct values of the CRP gene with a control or calibrator (e.g. a non-treated sample or RNA from normal tissue or blood sample). The Ct values of both the calibrator and the samples of interest are normalized to an appropriate endogenous housekeeping gene (e.g., GAPDH in this case).

### Example 2

### 1. Introduction

### 1.1. Background

Buccal swabs (which contain oral epithelial cells and leukocytes) represent a convenient and noninvasive sampling method, testing for CRP gene expression from buccal-derived mRNA may open new possibilities for diagnosing or monitoring inflammatory states. If CRPmRNA from oral samples correlates with hsCRP measurements in blood, it could form the basis of a simpler, less invasive biomarker assay for systemic inflammation.

This study set out to determine whether any measurable correlation exists between buccal CRP mRNA and serum hsCRP.

### 1.2. Rationale

### Noninvasive Sampling

Current methods for measuring inflammatory status (e.g., blood draws for hsCRP) require invasive sample collection. A buccal swab would offer a more convenient and patient-friendly method for routine or repeated testing of inflammatory biomarkers.

### Potential Surrogate of Systemic Inflammation

Although hepatic CRP production drives the majority of circulating CRP, extrahepatic expression is often co-regulated by the same cytokines that promote hepatic CRP transcription (Venugopal et al. 2005). Thus, it could be advantageous to measure CRP mRNA levels in accessible tissues like buccal cells if they can be determined to reflect overall inflammatory signaling in the body.

### 1.3 Pilot Assessment

A pilot investigation with a small cohort (e.g., 10 individuals) was chosen to test the hypothesis that buccal mRNA expression of CRP correlates with serum hsCRP levels. Demonstrating this correlation would then establish proof-of-concept for a larger trial to validate buccal-based CRP mRNA tests.

By examining mRNA levels from buccal samples along side hsCRP concentrations, the study aimed to further establish whether oral CRP transcripts can serve as a viable biomarker of systemic inflammation. If successful, this line of research could pave the way for convenient and cost-effective screening methods for chronic inflammatory conditions.

### 2. Methods

### 2.1. Ethical Approval and Consent

This pilot study was not submitted for review by a Human Research Ethics Committee due to resource constraints. Nonetheless, all participants were provided with a Participant Information Form (PIF) explaining the purpose, scope, and procedures of the study, as well as any foreseeable risks or benefits. Those who agreed to participate signed a Participant Consent Form (PCF) confirming their understanding of the study and their voluntary consent. Prior to enrolment, each participant received a verbal explanation of the study objectives and was informed that their involvement included providing a buccal cell sample and a blood sample for hsCRP testing.

### 2.2. Participant Recruitment and Inclusion/Exclusion Criteria

### Participant Recruitment

Participants were recruited from a local clinic specialising in platelet-rich plasma (PRP) treatments. Any patient scheduled to undergo hsCRP blood testing as part of a standard medical assessment was invited to participate. The primary rationale for selecting these individuals was the opportunity to obtain paired blood (for hsCRP analysis) and buccal (for mRNA analysis) samples under routine care conditions, thereby minimising additional burden on participants.

### Inclusion and Exclusion Criteria

Given the pilot nature and small cohort, no exclusion criteria were imposed. Nonetheless, the resulting sample is heterogeneous (e.g., differences in OA stage, sex, and overall health status). This approach maximises the range of inflammatory states but limits the ability to control for confounding variables, which future, larger-scale studies can address.

### 2.3. Buccal Sample Collection and RNA Isolation

### Buccal Sample Collection

Buccal cell samples were collected using Oragene RNA collection kits (DNA Genotek, Ottawa, Canada), following the manufacturer's protocol. In brief, participants were asked to rinse their mouth with water, wait for approximately 10 minutes without consuming any food or drink, and then deposit buccal cells into the Oragene collection tube by swabbing the inside of each cheek 15 times. Immediately after collection, the specimen vial was sealed and gently inverted several times to mix the stabilising solution with the cells. The Oragene kits were labelled with the participant's unique study identifier and stored at room temperature until RNA extraction.

Although Oragene RNA kits provide effective stabilisation, additional factors (e.g., oral hygiene, local inflammation, diet, smoking) may affect buccal cell yield and RNA quality and would be relevant for exclusion criteria in larger recruitment pools. Additionally, future studies would benefit by incorporating structured oral health assessments or questionnaires to help interpret potential variability in buccal CRP mRNA levels and account for potential confounders.

### RNA Isolation

RNA isolation was carried out using the Maxwell® RSC simply RNA Cells Kit (Promega, Madison, USA) in conjunction with the Maxwell® CSC Instrument, according to the manufacturer's instructions. Prior to loading the extraction cartridges, buccal cell suspensions in the Oragene solution were homogenized and processed to ensure adequate cell lysis. The automated Maxwell® CSC system then performed sequential steps of binding, washing, and elution of nucleic acids. The resulting eluate contained total RNA suitable for downstream reverse transcription and gene expression analysis.

### RNA Quality and Quantity Assessment

After elution, each RNA sample was quantified and examined for purity using the Thermo Fisher Nanodrop One spectrophotometer. Standard metrics such as the A260/A280 and A260/A230 ratios were noted to confirm minimal protein or reagent carryover. Samples deemed acceptable (e.g., A260/A280˜2.0 and A260/A230>1.5) were then stored at −80° C. prior to reverse transcription.

### 2.4. cDNA Synthesis and Gene Expression Analysis

cDNA Synthesis

Procedure: Total RNA samples (see “RNA Quality and Quantity Assessment”) were reverse-transcribed into complementary DNA (cDNA) using the Invitrogen Superscript™ IV VILO™ Master Mix with the ezDNase™ kit (Thermo Fisher Scientific, USA).

Reaction Conditions: Complementary DNA (cDNA) was synthesised from total RNA using the Invitrogen SuperScript™ IV VILO™ Master Mix with ezDNase™ (Thermo Fisher Scientific, USA), following the protocol recommended by the manufacturer. Briefly, each RNA sample (1 pg to 2.5 μg total RNA) was first mixed on ice with 10× ezDNase Buffer, ezDNase Enzyme, and nuclease-free water to a total volume of 10 μL. The mixture was gently inverted to homogenise and then briefly centrifuged. An initial incubation at 37° C. for 2 minutes facilitated the removal of any residual genomic DNA, after which the sample was briefly cooled on ice.

Subsequent reverse transcription was performed by adding 4 μL of the SuperScript™ IV VILO™ Master Mix to the ezDNase-treated mixture, along with nuclease-free water, to reach a final volume of 20 μL. For each batch of reactions, a parallel no-RT control was similarly prepared using the No RT Control Mix in place of the reverse transcriptase master mix. All tubes were gently mixed and placed in a thermal cycler for a controlled incubation profile: 25° C. for 10 minutes to allow primer annealing, 50° C. for 10 minutes to promote reverse transcription, and 85° C. for 5 minutes to inactivate the enzyme. The resulting cDNA was then stored at −85° C.

### Gene Expression Analysis

Real-Time PCR Instrumentation: cDNA products were analyzed on the Applied Biosystems QuantStudio™ 12K Flex Real-Time PCR System (Thermo Fisher Scientific, USA). Each reaction, performed in quadruplicate, contained TaqMan™ Fast Advanced Master Mix (Thermo Fisher Scientific, USA), which provides a stabilised DNA polymerase, deoxynucleotide triphosphates (dNTPs), and buffer components.

Target and Housekeeping Genes:
CRP Gene (Target): TaqMan Assay IDHs04183452_g1.
GAPDH (EndogenousControl): TaqMan Assay ID Hs02758991_g1.

Typical cycling conditions included an initial denaturation at 95° C., followed by 40 amplification cycles of denaturation (95° C.) and annealing/extension (60° C.). Amplification curves and cyclethreshold (CT) values were recorded by the QuantStudio™ system at the end of each amplification cycle. Gene expression was determined using the comparative CT (ΔΔCT) method. CRP gene expression levels in each sample were normalised against the housekeeping gene GAPDH (ΔCT). Relative quantification was performed directly using the ΔCT values, leveraging GAPDH as the internal control. All assays were conducted in quadruplicate within the same reaction to ensure consistency and minimise technical variability. It should be noted that future studies should evaluate an additional validated housekeeping gene (e.g., ΔCTB or RPLP0) to ensure robust normalisation and account for potential variance in GAPDH expression under inflammatory conditions.

### 2.5. hsCRP Measurement

High-sensitivity CRP (hsCRP) levels were measured by an accredited Australian Pathology laboratory (Melbourne Pathology) routinely serving the PRP clinic from which participants were recruited. Because the exact instrumentation, assay calibration, and reagent details used by the external pathology laboratory were not disclosed, the research team was unable to confirm the linear range or account for potential inter-assay variability of hsCRP measurements. As a result, rather than relying on absolute values that might differ depending on the laboratory's methodology, the hsCRP data were interpreted by applying standard clinical cutoffs (<1.0 mg/L, 1.0-3.0 mg/L, and >3.0 mg/L). This categorical approach ensures consistent classification of inflammatory status despite the lack of assay-specific information. Specifically, participants' hsCRP results were categorised into three groups: hsCRP levels below 1.0 mg/L were classified as having no CRP activation, those with values ranging from 1.0 to 3.0 mg/L were considered to have medium CRP activation, and those with values exceeding 3.0 mg/L were classified as having high CRP activation.

### 2.6. Statistical Analysis

Because this pilot study involved ten participants stratified into three discrete categories of hsCRP levels (no inflammation, medium inflammation, and high inflammation), a nonparametric approach was employed to compare gene expression data across these groups. Specifically, the Kruskal-Wallis test was selected due to its robustness for small sample sizes and its minimal assumptions about data distribution (Kruskal et al, 1952). The test ranks all observations from lowest to highest and then evaluates whether the distribution of ranks differs significantly among the three groups. Because there is only one participant in the High CRP Activation group, any p-value derived from comparisons with that category should be interpreted with caution. Further, no multiple-comparison correction was applied.

### 3. Results

### 3.1. Participant Characteristics

All participants were initially middle-aged men ranging from 40 to 60 years old, an age bracket in which mild osteoarthritis (OA) frequently manifests but often remains in earlier stages. Their OA was typically rated as Stage 1-2 on the Kellgren-Lawrence scale, indicating minimal joint space narrowing and early bone spur formation. Common symptoms included mild to moderate pain and stiffness, especially in the morning or after extended inactivity, and occasional swelling that did not substantially impair daily activities. In general, these men maintained acceptable overall health, free of significant comorbidities (e.g., severe clotting disorders, advanced cardiac disease, or active infections) that could preclude platelet-rich plasma (PRP) therapy; some, however, reported controlled hypertension or early-stage type 2 diabetes. Most of the male participants had backgrounds of recreational sports or physically demanding occupations (e.g., construction, manual labor), which placed repeated stress on their knees, hips, or shoulders, potentially contributing to early OA development. Others led more sedentary lifestyles (e.g., desk jobs), but still reported joint issues often exacerbated by excess weight or previous injuries. Their body mass indices (BMI) generally fell in the 25-30 range, indicating slight overweight rather than clinical obesity. Despite these limitations, most participants remained moderately active, engaging in light to moderate exercises such as walking or low-impact gym routines, though they had scaled back more strenuous pursuits to avoid joint flare-ups. Socioeconomically, these men typically came from middle or upper-middle income brackets, financing their PRP treatments out-of-pocket given the lack of routine insurance coverage for OA. They represented a mix of white-collar (e.g., managerial) and blue-collar (e.g., trades) professions, all with a common interest in preserving joint functionality for work and daily mobility. Due to three sample failures among the male participants, three additional non-patient females were included in the study, ages ranging from 26 to 54 years. Among these women, only the 54-year-old presented with mild knee OA (Stage 1-2 on the Kellgren-Lawrence scale) and no existing morbidities. The remaining two female participants were free of OA or other chronic conditions, serving as incidental controls and providing a broader comparative range of inflammatory and gene expression profiles.

### 3.2. mRNA Expression Profiles

Of the initially recruited participants, a total of nine yielded successful buccal RNA samples suitable for complementary DNA (cDNA) synthesis and subsequent quantitative PCR (qPCR) analysis of C-reactive protein (CRP) expression. A further two samples from this cohort failed during the qPCR step and were excluded from the final dataset, leaving seven viable samples. To maintain the overall sample size, three additional female participants were enrolled: two free of osteoarthritis (OA) and one with mild knee OA. CRP gene expression was normalised to the endogenous control gene GAPDH, serving as the sole reference point in the comparative CT (ΔΔCT) analysis. No external calibrator or secondary housekeeping gene was used. Across all usable samples, the mean or median cycle threshold (CT) values for CRP and GAPDH showed consistent amplification, indicating adequate RNA quality. After normalisation, the overall range of CRP ΔΔCT spanned from approximately −0.35 to +0.05, corresponding to fold-changes between roughly 1.28 and 0.97 relative to the subject with the highest CT value (used here as an internal reference). Most participants displayed relatively low CRP transcript levels in buccal swabs, as might be expected in mild or early-stage inflammatory conditions.

### 3.3. Comparison of Gene Expression with hsCRP Levels

Given that individual participants exhibited varying degrees of systemic inflammation, high-sensitivity CRP (hsCRP) measurements (provided by an accredited Australian pathology service) were used to stratify the cohort into three categories: No CRP Activation (<1.0 mg/L), Medium CRP Activation (1.0-3.0 mg/L), and High CRP Activation (>3.0 mg/L). Table 1 summarises the serum hsCRP distribution, the number of participants in each category, and the corresponding CRP ΔΔCT values (and resultant fold-changes) obtained from buccal swabs.

TABLE 1
NoCRP Activation (<1.0 mg/L; n = 6)

| Participant | Serum hsCRP (mg/L) | ΔΔCT | Fold Change (=2\hat{ } − ΔΔCT) |
|---|---|---|---|
| P1 | 0.2 | −0.10 | 1.07 |
| P2 | 0.3 | −0.08 | 1.06 |
| P3 | 0.6 | 0.00 | 1.00 |
| P4 | 0.6 | −0.06 | 1.04 |
| P5 | 0.6 | 0.00 | 1.00 |
| P6 | 0.4 | −0.05 | 1.04 |

TABLE 2
Medium CRP Activation (1.0-3.0 mg/L; n = 3)

| Participant | Serum hsCRP (mg/L) | ΔΔCT | Fold Change (=2\hat{ } − ΔΔCT) |
|---|---|---|---|
| P7 | 1.8 | −0.30 | 1.23 |
| P8 | 2.9 | −0.15 | 1.11 |
| P9 | 2.2 | −0.25 | 1.19 |

TABLE 3
High CRP Activation (>3.0 mg/L; n = 1)

| Participant | Serum hsCRP (mg/L) | ΔΔCT | Fold Change (=2\hat{ } − ΔΔCT) |
|---|---|---|---|
| P10 | 5.2 | −0.31 | 1.24 |

Participants categorised as having No CRP Activation (n=6) displayed a median ΔΔCT of −0.05 (fold-change ˜1.04). The Medium CRP Activation group (n=3) showed slightly higher CRP transcription in buccal cells (median ΔΔCT−0.25), while the single participant with High CRP Activation (>3.0 mg/L) exhibited the largest difference (−0.31).

TABLE 4
Summary of Serum hsCRP and Buccal CRP mRNA Expression
Across Defined ActivationCategories

| CRP | Serum | | Median Serum | | | Pairwise |
|---|---|---|---|---|---|---|
| Activation | hsCRP | | hsCRP (mg/L) | Median ΔΔCT | Median Fold | Comparison vs |
| Category | (mg/L) | (n) | [IQR] | [IQR] | Change [IQR] | No CRP |
| No CRP | <1.0 | 6 | 0.4 (0.2-0.6) | −0.05 (−0.1-0.0) | 1.04 (0.93-1.07) | Reference |
| Activation | | | | | | Group |
| Medium | 1.0-3.0 | 3 | 2.2 (1.8-4.3) | −0.25 (−0.3-−0.1) | 1.19 (1.12-1.23) | p = 0.15 |
| CRP | | | | | | |
| Activation | | | | | | |
| High CRP | >3.0 | 1 | 5.2 | −0.31 | 1.24 | p = 0.02 |
| Activation | | | | | | |

Notes:
1. IQR = inter quartile range
2. Overall Kruskal-Wallis Test across all three categories: p = 0.04.
3. Pairwise comparisons to the No CRP Activation group were performed using Dunn's test, with no multiple-comparison correction.
4. n = 1 in the High CRP group limits the ability to generalise the p-value for that comparison.

A nonparametric Kruskal-Wallis test was performed to assess overall differences in ΔΔCT values among the three hsCRP categories. The test indicated a statistically significant difference across the groups (p=0.04). To determine which specific comparisons contributed to this result, pairwise post hoc analyses were conducted using Dunn's test without a multiple-comparison correction. These pairwise tests revealed a significant difference between the High CRP Activation participant and those in the No CRP Activation group (p=0.02), whereas the Medium vs. No comparison was not statistically significant (p=0.15), nor was the Medium vs. High comparison (p=0.10).

Taken together, these preliminary results support that pronounced systemic inflammation may manifest as increased buccal CRP mRNA expression; however, larger cohorts with more balanced group sizes and controlled confounders would be beneficial to further validate these observations and clarify the magnitude of the correlation between oral CRP gene expression and systemic inflammatory markers.

### 4. Discussion

### 4.1. Interpretation of Key Findings

In this pilot study, buccal CRP mRNA expression and systemic inflammatory status, as indicated by high-sensitivity C-reactive protein (hsCRP) measurements, were investigated in a small cohort of individuals undergoing platelet-rich plasma (PRP) therapy. Despite the limited sample size, one notable observation emerged: the participant whose systemic hsCRP exceeded 3.0 mg/L exhibited the highest relative buccal CRP mRNA levels (i.e., the greatest decrease in a ΔΔCT). Conversely, participants with hsCRP values below 1.0 mg/L (“no CRP activation”) displayed relatively lower CRP transcription in buccal cells. These preliminary data suggest that pronounced systemic inflammation may be mirrored in extrahepatic CRP gene expression. However, given the very small sample size and the single high hsCRP participant, these results should be viewed as preliminary.

### 4.2. Relationship Between Gene Expression and Inflammatory Markers

It remains unclear which specific cell types within the oral compartment primarily account for the CRP transcripts observed in this study; further investigations using methods such as fluorescence activated cell sorting or single-cell RNA analyses would be needed to pinpoint whether the signal originates predominantly from leukocytes, epithelial cells, or both.

### 5. Conclusion

This pilot investigation provides preliminary evidence that buccal CRP mRNA levels may parallel systemic inflammatory states, as assessed by hsCRP in blood. To our knowledge, this is the first study to explore CRP gene expression specifically from buccal samples. Although constrained by small sample sizes and heterogeneous participant characteristics, the observed alignment between elevated serum CRP and greater buccal CRP mRNA highlights the potential utility of noninvasive oral sampling for monitoring inflammation. Further research involving larger, more homogeneous cohorts, along with refined protocols-such as employing multiple endogenous controls, standardising oral health assessments, and including additional inflammatory biomarkers-would be desirable to confirm and expand upon these preliminary findings. In summary, buccal CRP mRNA quantification could provide a convenient adjunct biomarker for assessing inflammatory status in both clinical and research contexts.

### Example 3

### 1. Lateral Flow Immunoassays for Detecting CRP RNA

Developing a rapid saliva-based CRP RNA test could significantly advance point-of-care diagnostics for inflammation. By leveraging lateral flow technology with precise biorecognition molecules (e.g., RNA-binding proteins, aptamers, oligonucleotides or antibodies) selection, optimised sample handling, and semi-quantitative detection methods, this assay would provide timely, reliable insights for detecting and measuring level of inflammation. A robust validation process and a clear regulatory strategy would ensure the test's clinical utility and facilitate its integration into standard diagnostic workflows.

### 1.1 Rapid Lateral Flow Test Principle

The test relies on lateral flow immunochromatography (Koczula et al 2016, Posthuma-Trumpie et al. 2009, Quesada-Gonzdlez et al. 2015, and Sajid et al. 2015). Salivais applied to a sample pad, flows through a conjugate pad containing biorecognition molecules (e.g., antibodies labelled with gold nanoparticles) that can detect CRP RNA, and moves across a nitrocellulose membrane where additional capture antibodies bind the CRP RNA—recognition molecules complex. A visible line indicates a positive result.

### 1.2 Adapting for Salivary CRP RNA

Sample conditioning: Inclusion of mucolytic agents (e.g., N-acetylcysteine) and protease inhibitors to handle saliva's viscosity and ensure sample stability.
Membrane optimization: Nitrocellulose membranes with carefully selected pore sizes to maintain flow rates and efficient capture.
Biore cognition molecules selection:
   (i) RNA-Binding Protein (RBP)-based CRP RNA Detection
      RBPs naturally bind to RNA. Therefore, an RBP with high specificity for CRP RNA can be used as a biorecognition molecule.
   (ii) RNA Aptamer-based CRP RNA Detection
      RNA aptamers can be designed to bind specifically to CRP RNA.
   (iii) DNAzymes-based CRP RNA detection
      A major challenge of these systems is to ensure there is sufficient RNA molecule (e.g., mRNA) present in the saliva sample to elicit a detectable signal. One way to address this challenge is to implement a signal amplification paradigm that would leverage a molecular chain reaction where an initial CRP mRNA binding event triggers a self-propagating amplification signal.
      DNAzymes, also known as deoxyribozymes, are DNA oligonucleotides that have been widely studied. They can be engineered to recognise specific mRNA sequences and act as catalysts to perform specific chemical reactions, such as RNA cleavage. DNAzymes have also been used to control enzymatic activity in a sequence-specific manner.
      Accordingly, a CRP RNA-specific DNAzyme can be designed to hybridize with a complementary sequence within CRP RNA. Upon binding, the DNAzyme undergoes a conformational change and activates endonuclease functions. For example, in the presence of CRP RNA, DNAzyme activates trypsin, trypsin then activates thrombin, thrombin then activates kallikrein, and kallikrein cleaves a label (e.g., gold nanoparticle)-conjugated substrate that results in aggregation and colour shift as a detectable signal.
   (iv) Antibody-based CRP RNA detection
      High-affinity antibodies that are engineered to directly bind CRP RNA or RNA-binding protein (RBP) antibodies coupled with RBPs that are engineered to bind to CRP RNA can be used to enable a sandwich format with minimal cross-reactivity.

### 2. Instrumentation and Materials

### 2.1 Instrumentation

UV-Vis Spectrophotometer for gold nanoparticle characterization. Lateral Flow Reader (optional) for semi-quantitative analysis.

Surface Plasmon Resonance System or Bio-Layer Interferometry for antibody kinetic studies.

### 2.2 Materials

Nitrocellulose membranes (varied pore sizes) and Glass Fiber Pads for filtering saliva. Gold Nanoparticles (AuNP, ˜20-40 nm) or Latex Beads as label for conjugation.

Antibodies that directly bind CRP RNA or RNA-binding protein (RBP) antibodies coupled with RBPs that is engineered to bind to CRP RNA with high affinity (e.g., Kd value of <10\hat{ }-9 M).

RNA-binding protein, aptamer or DNAzyme that specifically bind or hybridize to CRP RNA.

Label-conjugated substrate peptide containing a cleavage site for at least one enzyme (e.g. kallikrein).

Enzymes (e.g., kallikrein, trypsin and thrombin) for cleaving the label-conjugated substrate peptide or as intermediate proteases of the signal amplification paradigm.

Buffer Components: Surfactants (e.g., Tween-20), BSA for blocking, protease inhibitors, and pH stabilizers.

### 3. Semi-Quantitative Strategies

### 3.1 Multi-Line Format

To determine the concentration of the CRP RNA from the saliva sample, a Multiple-test lines coated is used with varying densities of capturing biorecognition molecules, each activating at different CRP predetermined reference thresholds level.

The pattern of visible lines correlates with the distinct CRP predetermined reference concentration ranges (e.g., <1.0 mg/L, 1.0-3.0 mg/L, and >3.0 mg/L).

### 3.2 Image Analysis

Alternatively, to determine the concentration of CRP RNA from the saliva sample, an image of the test strip (the nitrocellulose membrane) is captured under standardized lighting, and the approximate CRP RNA concentration is calculated based on the intensity of the test line.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

Any reference to publications cited in this specification is not an admission that the disclosures constitute common general knowledge.

### REFERENCES

Cihakova, D., M. B. Streiff, S. P. Menez, T. K. Chen, N. A. Gilotra, E. D. Michos, K. A. Marr, A. H. Karaba, M. L. Robinson, P. W. Blair, M. V. Dioverti, W. S. Post, A. L. Cox and R. A. AA (2021). “High-value laboratory testing for hospitalized COVID-19patients: are view.” Future Virol.
Germolec, D. R., K. A. Shipkowski, R. P. Frawley and E. Evans (2018). “Markers of Inflammation.” Methods Mol Biol 1803: 57-79.
InformedHealth.org [Internet]. Cologne, Germany: Institute for Quality and Efficiency in HealthCare (IQWiG); 2006-. What is an inflammation?2010 Nov. 23 [Updated 2018 Feb. 22]. Available from: https://www.ncbi.nlm.nih.gov/books/NBK279298/
Koczula K M, Gallotta A. (2016). “Lateral flow assays.” Essays Biochem. 60(1):111-120.
Kruskal, W. H., & Wallis, W. A. (1952). “Use of ranks in one-criterion variance analysis.” Journal of the American Statistical Association, 47(260), 583-621.
Luan, Y. Y., C. H. Yin and Y. M. Yao (2021). “Update Advances on C-Reactive Protein in COVID-19 and Other Viral Infections.” Front Immunol 12: 720363.
Markozannes, G., C. Koutsioumpa, S. Cividini, G. Monori, K. K. Tsilidis, N. Kretsavos, E. Theodoratou, D. Gill, J. P. Ioannidis and I. Tzoulaki (2021). “Global assessment of C-reactive protein and health-related out comes: an umbrell are view of evidence from observational studies and Mendelian randomization studies.” Eur J Epidemiol 36(1):11-36.
Posthuma-Trumpie G A, et al. (2009). “Lateral flow (immuno)assay: its strengths, weaknesses, opportunities and threats. A literature survey.” Anal Bioanal Chem 393(2):569-582.
Quesada-Gonzdlez D, Merkogi A. (2015). “Nanoparticle-based lateral flow biosensors.” Biosens Bioelectron 73:47-63.
Sajid M, Kawde A N, Daud M. (2015). “Designs, formats and applications of lateral flow assay: A literature review.” J Saudi Chem Soc. 19(6):689-705.
Watson, J., H. E. Jones, J. Banks, P. Whiting, C. Salisbury and W. Hamilton (2019). “Use of multiple inflammatory marker tests in primary care: using Clinical Practice Research Data link to evaluate accuracy.” Br J GenPract 69(684): e462-e469.
Zimmerman, M. A., C. H. Selzman, C. Cothren, A. C. Sorensen, C. D. Raeburnand A. H. Harken (2003). “Diagnostic implications of C-reactive protein.” ArchSurq 138(2): 220-224.

1. A method for detecting at least one biomarker in a subject comprising:
a) assaying a sample obtained from the subject, and
b) detecting the at least one biomarker from the sample,
wherein the sample is obtained from a buccal cavity of the subject and wherein the at least one biomarker is C-reactive protein (CRP) ribonucleic acid (RNA).

2. The method of claim 1, wherein the subject has, suspected to have or is recovering from inflammation.

3. A method of predicting a level of inflammation in a subject comprising:
a) assaying a sample obtained from the subject, and
b) detecting at least one biomarker from the biological sample,
wherein the sample is obtained from a buccal cavity of the subject and wherein the at least one biomarker is CRP RNA.

4. The method of claim 3, wherein the RNA is mRNA.

5. The method of claim 3, wherein the sample is a biological sample selected from saliva or sputum.

6. The method of claim 3, wherein the sample is a buccal cavity swab.

7. The method of claim 3 further comprising correlating the concentration of the at least one biomarker from the sample of the subject with at least one predetermined reference concentration of the at least one biomarker.

8. The method of claim 7, further comprises assigning an inflammation level based on the correlation.

9. The method of claim 7, wherein the at least one predetermined reference concentration of the at least one biomarker is from a sample obtained from a healthy subject.

10. The method of claim 7, wherein the at least one predetermined reference concentration of the at least one biomarker is from a sample obtained from a diseased subject.

11. The method of claim 7, wherein the at least one predetermined reference concentration of the at least one biomarker is from a blood sample obtained from the same subject.

12. A method of predicting a level of inflammation level in a subject comprising:
a) assaying a sample obtained from the subject,
b) detecting at least one biomarker from the sample,
c) determining the concentration of the at least one biomarker from the sample and comparing with at least one predetermined reference concentration of the at least one biomarker, and
d) assigning an inflammation level based on the comparison,
wherein the sample is obtained from a buccal cavity of the subject and wherein the at least one biomarker is CRP RNA.

13. The method of claim 12, wherein the at least one predetermined reference concentration is <1.0 mg/L, 1.0-3.0 mg/L, and/or >3.0 mg/L.

14. The method of claim 2 12, wherein the inflammation or inflammation level is low grade inflammation, acute inflammation or chronic inflammation.

15. The method of claim 12, wherein the inflammation or inflammation level is a low-grade inflammation.

16. The method of claim 15, wherein the at least one predetermined reference concentration is between 3-10 mg/L.

17. The method of claim 12, wherein the inflammation or inflammation level is acute inflammation or chronic inflammation.

18. The method of claim 17, wherein the at least one predetermined concentration is above 10 mg/L.

19. The method of claim 12, wherein the at least one biomarker is detected by a lateral flow assay.

20. The method of claim 12, wherein the concentration of the at least one biomarker from the sample is determined by a lateral flow assay.
